(19) United States
(12) Patent Application Publication
(10) Pub. No.: US 2008/0300495 A1
(43) Pub. Date: Dec. 4, 2008

(54) Method of eliminating vibration interferences of a sphygmomanometer for measuring blood pressure

(75) Inventors: Paul Yang, Taipei (TW); Jung-Chi Chang, Yunlin (TW)

Correspondence Address: BACON & THOMAS, PLLC, 625 SLATERS LANE, FOURTH FLOOR, ALEXANDRIA, VA, 22314-1176, US

(73) Assignee: HEALTH & LIFE CO., LTD, Chung Ho City (TW)

(21) Appl. No.: 11/802,949

(22) Filed: May 29, 2007

(51) Int. Cl. A61B 5/00

(52) U.S. Cl. 600/494

(57) ABSTRACT

A method of eliminating vibration interferences of a sphygmomanometer for measuring blood pressure uses a pressure sensor and an acceleration sensor to measure a voltage-time variation waveform of blood pressure detected by an acceleration sensor. An interference variation waveform is subtracted from the measured voltage-time variation waveform to calculate the correct blood pressure value. During measurement, the acceleration sensor detects and determines whether the vibration interference falls within a predetermined allowable range; if yes, a warning signal is issued to remind users to measure blood pressure again; if no, the acceleration sensor detects an interference variation waveform. The inference variation waveform obtained by a conversion function is subtracted from the voltage-time variation waveform when the blood pressure measurement is affected by external vibration interferences, and the original voltage-time variation waveform not affected by vibration interferences is restored, so as to measure a correct blood pressure value.

### BACKGROUND OF THE INVENTION

1. Field of the Invention

The present invention relates to a method of measuring blood pressure, and more particularly to a method of measuring blood pressure that subtracts an inference variation waveform obtained by a conversion function from a voltage-time variation waveform of blood pressure and pulse when the measurement of the blood pressure is affected by external vibration interferences and restores the original voltage-time variation waveform that is not affected by vibration interferences and measures a correct blood pressure value.

2. Description of the Related Art

In general, sphygmomanometers are divided mainly into traditional mercury sphygmomanometers and electronic sphygmomanometers. The mercury sphygmomanometers not only take much time for the measurement, but also require professionally trained and experienced people to measure a correct blood pressure. On the other hand, electronic sphygmomanometers are divided into desktop, wrist, tunnel and finger sphygmomanometers. Since the electronic sphygmomanometers do not require any pre-adjustment or pre-correction procedure, and the results of measurement are displayed by a display device, therefore the application is very simple and easy. Although electronic sphygmomanometers are very convenient to use, the precision of measurements on only relates to the time of a day (such as a lower blood pressure in the morning and a higher blood pressure at noon), but also relates to the condition whether or not the wrist or a related part of the user's body is shaken and the disposing position of the sphygmomanometer. For instance, a correct way of measuring blood pressure by a wrist sphygmomanometer is to place the measuring device of the sphygmomanometer at the position level with a user's heart, and measure the blood pressure while the wrist and the sphygmomanometer are being held still. However, users may move or shake their wrists or the sphygmomanometer during a blood pressure measurement for different reasons and such movement or shaking may cause interferences to the sphygmomanometer, and thus the blood pressure cannot be measured accurately.

### SUMMARY OF THE INVENTION

In view of the foregoing shortcomings of the prior art, the inventor of the present invention based on years of experience in the related industry to conduct extensive researches and experiments, and finally developed a method of eliminating vibration interferences of a sphygmomanometer for measuring blood pressure in accordance with the present invention.

The primary objective of the present invention is to provide a method of eliminating vibration interferences of a sphygmomanometer for measuring blood pressure, and the method subtracts an error occurred in a blood pressure measurement due to vibration interferences and calculates a correct blood pressure value. Another objective of the present invention is to provide a method of using a pressure sensor and an acceleration sensor of a sphygmomanometer to detect a user's blood pressure and pulse when vibrations occur during the measurement of blood pressure and a voltage-time variation waveform of the acceleration sensor, and the interference waveform variation caused by vibration is subtracted from the measured voltage-time variation waveform of the blood pressure and pulse to calculate a correct blood pressure value.

A further objective of the present invention is to provide a method of eliminating vibration interferences of a sphygmomanometer for measuring blood pressure, wherein an acceleration sensor in the sphygmomanometer detects whether of not the measured vibration interference falls within an allowable range, and issues a warning signal to remind the user to take the measurement again, if the detected vibration interference exceeds the allowable range.

Another further objective of the present invention is to issue a warning signal through a display device (such as an LCD screen or an LED indicating lamp) or a speaker, and the warning signal can be displayed in the form of voice, music, sound, text in different colors, word or figure.

### BRIEF DESCRIPTION OF THE DRAWINGS

FIG. 1 is a flow chart of measuring blood pressure in accordance with the present invention;

FIG. 2 is a schematic block diagram of a logic circuit of the present invention;

FIG. 3 shows a voltage-time waveform variation detected by an acceleration sensor and a pressure sensor when vibration interferences occur; and

FIGS. 4 and 4A are schematic views of the operation of a wrist sphygmomanometer in accordance with the present invention.

### DETAILED DESCRIPTION OF THE PREFERRED EMBODIMENTS

To make it easier for our examiner to understand the present invention, the following detailed description with reference to the accompanying drawings of a preferred embodiment is given for example, but such preferred embodiment is not intended to limit the scope of the present invention. For simplicity, like numerals are used for like elements for the description of the specification of the present invention.

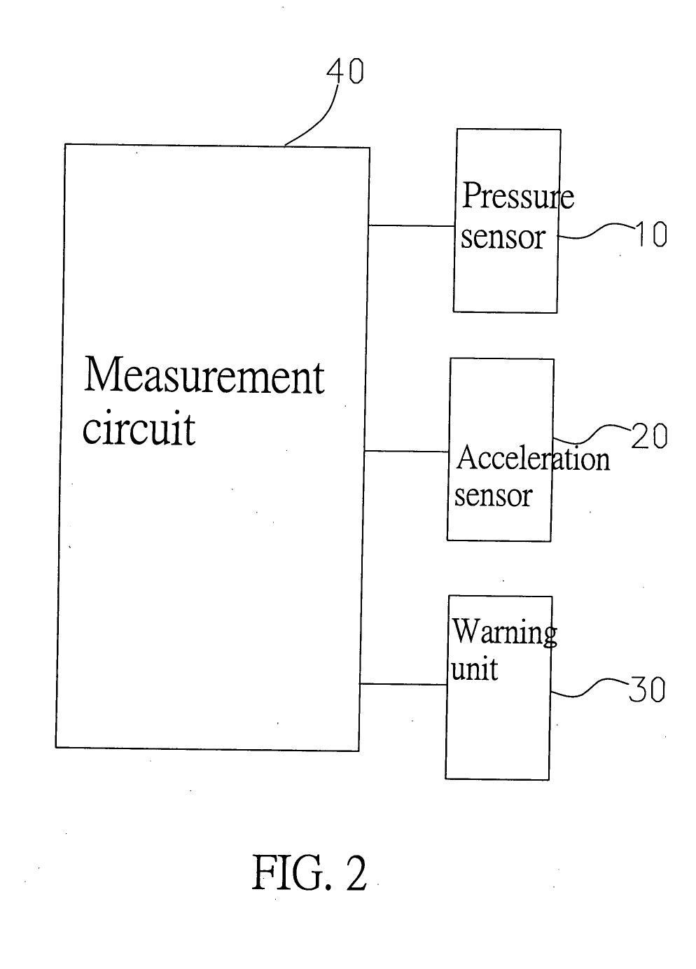

Referring to FIG. 2 for a method of eliminating vibration interferences of a sphygmomanometer for measuring blood pressure, the sphygmomanometer is comprised of a pressure sensor 10, an acceleration sensor 20, a warning unit 30 and a measurement circuit 40, and the pressure sensor 10, the acceleration sensor 20, the warning unit 30 are connected with the measurement circuit 40 (which is the same as the general blood pressure measurement circuit, and thus their structures will not be described in detail here); wherein the pressure sensor 10 uses a pressure difference of the blood pressure and pulse to detect a voltage-time variation waveform.

The acceleration sensor 20 of a preferred embodiment has an acceleration detection chip (but the ordinary person skilled in the art can use another component such as an acceleration sensor to detect two-dimensional spatial variations or three-dimensional spatial variations) for producing different voltage variations by a spatial position variation and detecting a voltage-time variation waveform when the detector is interfered by vibrations.

The warning unit 30 can be an LCD screen, a LED indicating lamp or a speaker, so that when the vibration interference exceeds an allowable ranges during a blood pressure measurement, voices, music, sounds, lamplights of different colors, figures or words are used for reminding users.

The measurement circuit 40 receives voltage-time variation waveforms detected by the pressure sensor 10 and the acceleration sensor 20 for the calculation.

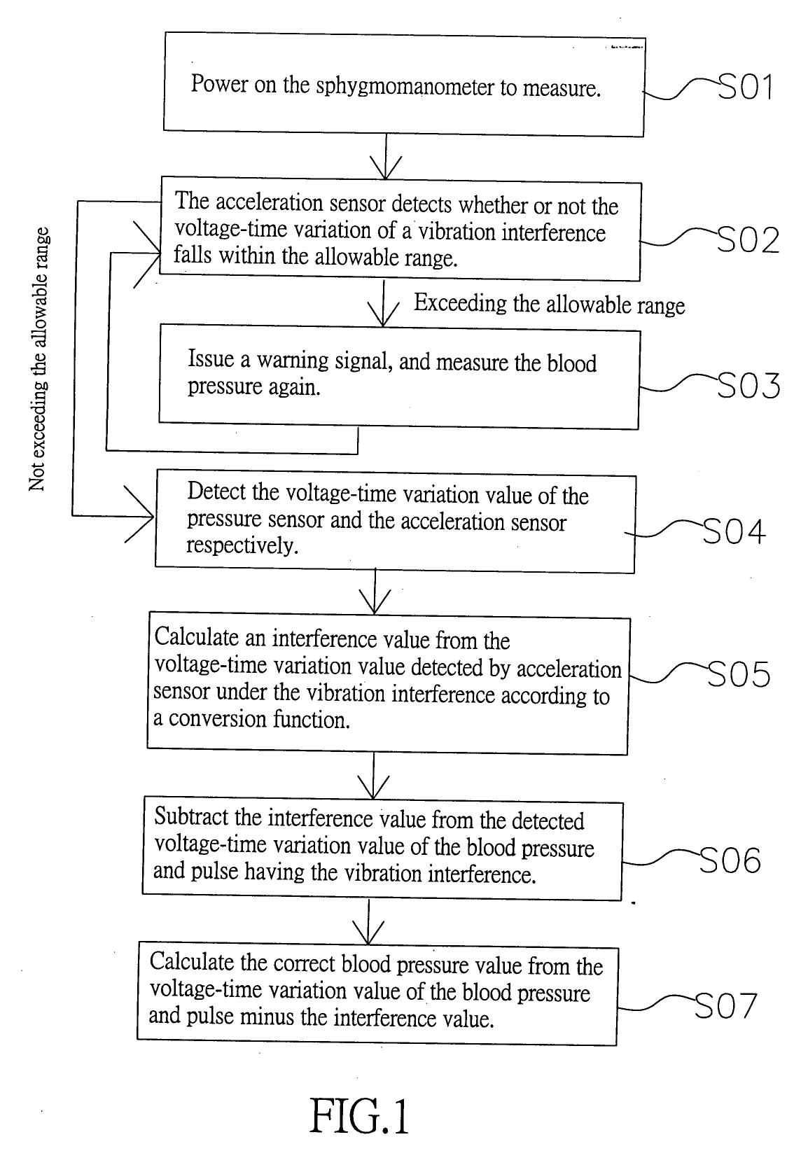

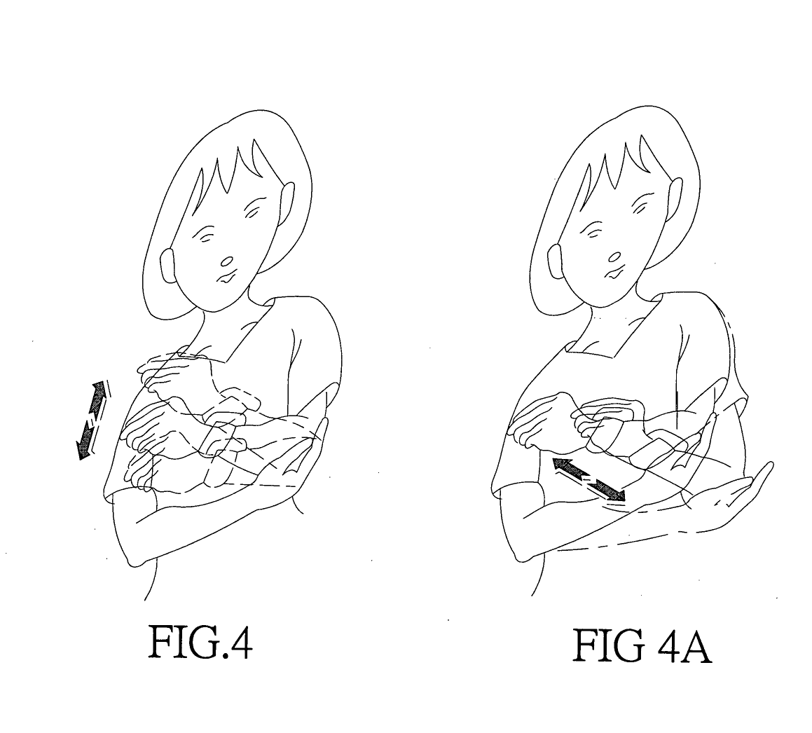

Referring to FIGS. 1, 2, 4 and 4A, this embodiment uses an electronic wrist sphygmomanometer for the illustration, and its related technical contents, characteristics and performance of the present invention are described as follows. The method of eliminating vibration interferences of a sphygmomanometer for measuring blood pressure comprises the steps of:

Step S01: powering on a sphygmomanometer to execute the function of measuring blood pressure;

Step S02: determining whether or not a vibration interference detected by an acceleration sensor 20 of the sphygmomanometer falls within an allowable range, and, executing Step S03, if the vibration interference exceeds the allowable range, or else executing Step S04;

Step S03: issuing a warning signal by a warning unit 30 to remind users, and executing Step S02 again, and the warning signal of this embodiment can be voice, music, sound, lamplight of different colors, figure or word, and the display device can be LCD screen, LED screen, lamplight or speaker);

Step S04: detecting voltage-time variation waveforms by a pressure sensor 10 and an acceleration sensor 20 respectively, and executing Step S05;

Step S05: calculating an interference waveform variation from a voltage-time variation waveform detected by an acceleration sensor 20 during vibration interferences according to a conversion function, and executing Step S06;

Step S06: subtracting an interference waveform variation from the detected voltage-time variation waveform of the blood pressure and pulse during vibration interferences, and executing Step S07; and

Step S07: calculating the correct blood pressure value by the voltage-time variation waveform of the blood pressure and pulse minus the interference waveform variation.

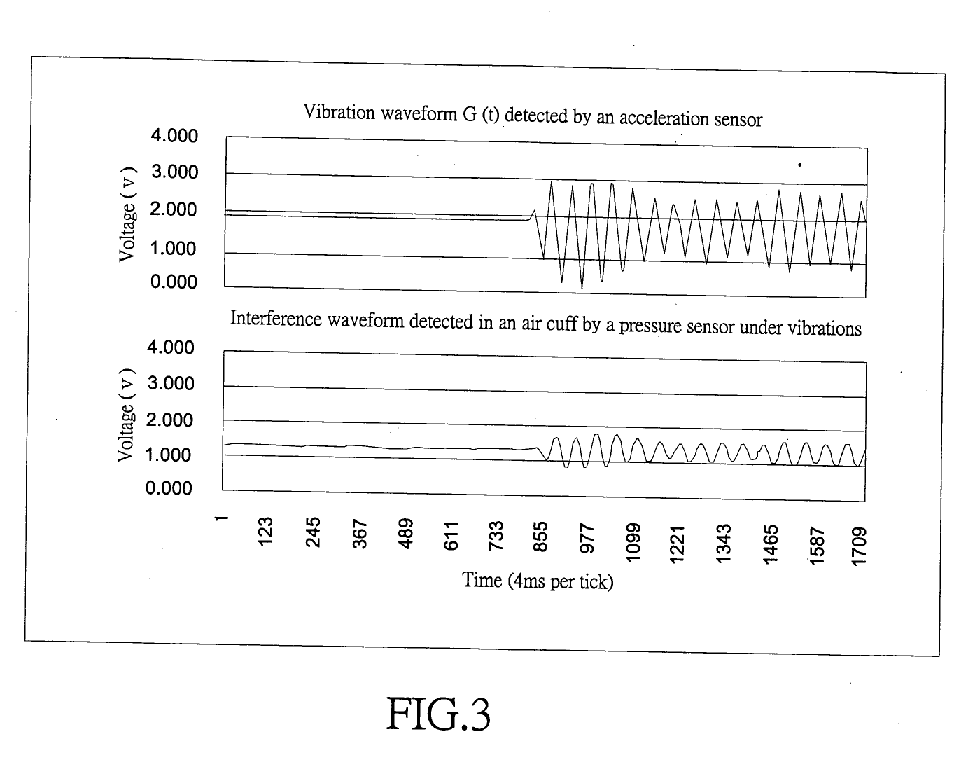

In FIG. 3, the method of deriving the conversion function of Step S05 is described, and an electronic wrist sphygmomanometer is used for illustration. The sphygmomanometer includes a pressure sensor, an acceleration sensor and an air cuff worn around a user's wrist, and a dummy without blood pressure is used for the measurement. The sphygmomanometer is shaken, such that when a blood pressure is measured by the pressure sensor and the acceleration sensor, the air cuff is inflated and vibrated, and the voltage-time variation waveform detected by the pressure sensor and the voltage-time variation waveform detected by the acceleration sensor of the sphygmomanometer under vibrations are produced. After the measurement, the upper half of the figure shows the voltage-time variation waveform detected by the acceleration sensor when the acceleration sensor is shaken, and the lower half of the figure is the voltage-time variation waveform detected by the pressure sensor when it is interfered. With a careful review of waveforms at the upper and lower halves of the figure, we can easily observe that the number of cycles is the same. In the meantime, the voltages of the pressure sensor and the acceleration sensor are in a relation of one-to-one correspondence. In other words, the voltage measured by the pressure sensor (which is simply the interference waveform variation under the interference of vibrations) can be used to derive the conversion function (such as F(Δt)) statistically from the voltage detected by the acceleration sensor, and thus the method of eliminating the vibration interferences can be explained statistically by a corresponding mathematical function given below:

P″(Δt)=P(Δt)+G(Δt)*F(Δt)

Where, P″ (Δt) is the voltage-time variation waveform detected by the pressure sensor in actual measurements, including the voltage-time variation waveforms of the blood pressure and vibration interferences; P(Δt) is the voltage-time variation waveform of the human blood pressure and pulse detected by pressure sensor under the ideal condition without being affected by vibrations; G(Δt) is the voltage-time variation waveform detected by the acceleration sensor under vibrations; F(Δt) is the conversion function of the aforementioned conditions, and the voltage detected by the acceleration sensor is multiplied with the conversion function to obtain the interference waveform variation of the pressure sensor; G(Δt)*F(Δt) is the interference waveform variation of the pressure sensor; and (Δt) is the measured time (which is the time difference within the same time). Thus, the following equation can be obtained.

P(Δt)=P″(Δt)−G(Δt)*F(Δt)

In other words, the correct voltage-time variation waveform of the pressure sensor without vibrations is equal to the voltage-time variation waveform of the vibration interferences minus the product of the voltage-time variation waveform obtained from the conversion function and detected by the acceleration sensor.

In summation of the description above, the present invention can be applied in various different electronic sphygmomanometers such as desktop, wrist, tunnel and finger sphygmomanometers, and it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of eliminating vibration interferences of a sphygmomanometer for measuring blood pressure, and the method utilizes a pressure sensor, an acceleration sensor and a warning unit in the sphygmomanometer to measure a correct blood pressure value when the measurement is taken under vibrations, and the method comprising the steps of:
Step S01: powering on the sphygmomanometer to execute the function of measuring blood pressure;
Step S02: detecting whether or not a vibration interference detected by the acceleration sensor of the sphygmomanometer falls within an allowable range, and executing Step S03 if the detected vibration interference falls within the allowable range, or else executing Step S04;
Step S03: issuing a warning signal to remind a user to execute Step S02 again;
Step S04: detecting voltage-time variation waveforms of the pressure sensor and the acceleration sensor respectively, and executing Step S05;
Step S05: calculating an interference waveform variation from the voltage-time variation waveform detected by acceleration sensor under the vibration interference according to a conversion function, and executing Step S06;
Step S06: subtracting the interference waveform variation from the detected voltage-time variation waveform of the blood pressure and pulse having the vibration interference, and executing Step S07; and
Step S07: calculating the correct blood pressure value from the voltage-time variation waveform of the blood pressure and pulse minus the interference waveform variation.

2. The method of eliminating vibration interferences of a sphygmomanometer for measuring blood pressure as recited in claim 1, wherein the pressure sensor detects the voltage-time variation waveform by a pressure difference of the blood pressure and pulse.

3. The method of eliminating vibration interferences of a sphygmomanometer for measuring blood pressure as recited in claim 1, wherein the acceleration sensor produces a voltage variation by spatial position variation of different angles for detecting a voltage-time variation waveform, when a vibration interference occurs.

4. The method of eliminating vibration interferences of a sphygmomanometer for measuring blood pressure as recited in claim 3, wherein the acceleration sensor detects a two-dimensional space variation.

5. The method of eliminating vibration interferences of a sphygmomanometer for measuring blood pressure as recited in claim 3, wherein the acceleration sensor detects a three-dimensional space variation.

6. The method of eliminating vibration interferences of a sphygmomanometer for measuring blood pressure as recited in claim 1, wherein the warning unit is an LCD, a LED screen, a lamplight or a speaker.

7. The method of eliminating vibration interferences of a sphygmomanometer for measuring blood pressure as recited in claim 6, wherein the warning signal issued by the warning unit is voice, music, sound, sentence of different colors, word, lamplight or figure.
